(19) United States
(12) Patent Application Publication
(10) Pub. No.: US 2026/0218152 A1
(43) Pub. Date: Jul. 30, 2026

(54) CRISPR-CAS SYSTEM USING MOLECULAR CHAPERONE TO ENHANCE TRANS-CLEAVAGE ACTIVITY AND APPLICATION THEREOF

(71) Applicant: Innovation Academy for Precision Measurement Science and Technology, Chinese Academy of Sciences, Wuhan (CN)

(72) Inventors: Lichun HE, Wuhan (CN); Yihao CHEN, Wuhan (CN); Xu ZHANG, Wuhan (CN); Maili LIU, Wuhan (CN)

(73) Assignee: Innovation Academy for Precision Measurement Science and Technology, Chinese Academy of Sciences, Wuhan (CN)

(21) Appl. No.: 19/464,530

(22) Filed: Jan. 29, 2026

Related U.S. Application Data
(63) Continuation of application No. PCT/CN2025/084421, filed on Mar. 24, 2025.

(30) Foreign Application Priority Data
Jan. 27, 2025 (CN) 202510125843.2

(51) Int. Cl. C12N 9/22; C07K 14/00; C07K 14/195; C07K 14/47; C12N 15/11

(52) U.S. Cl. CPC C12N 9/226; C07K 14/00; C07K 14/195; C07K 14/47; C12N 15/11; C12N 2310/20; C12N 2320/10

(57) ABSTRACT

A CRISPR-Cas system using a molecular chaperone to enhance trans-cleavage activity and an application thereof are provided. The CRISPR-Cas system includes: 1 pM-1 mM Cas enzyme, 1 pM-1 mM crRNA, 1*reaction buffer, 0.1 pM-1 mM target substrate, 1 nM-1 mM ssDNA reporter gene, and 1 pM-1 mM molecular chaperone. The above design adds a molecular chaperone selected from ClpB, HSP70, Spy, or HSP20 to the CRISPR-Cas system, thereby increasing the trans-cleavage activity of the Cas enzyme to different extents and enhancing detection sensitivity.

### CROSS REFERENCE TO THE RELATED APPLICATIONS

This application is a continuation application of International Application No. PCT/CN2025/084421, filed on Mar. 24, 2025, which is based upon and claims priority to Chinese Patent Application No. 202510125843.2, filed on Jan. 27, 2025, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named GBWHZQ013_Sequence_Listing.xml, created on 01/19/2026, and is 6,387 bytes in size.

### TECHNICAL FIELD

The present invention relates to the technical field of gene editing and molecular diagnostics, and in particular to a CRISPR-Cas system using a molecular chaperone to enhance trans-cleavage activity and an application thereof.

### BACKGROUND

The CRISPR-Cas system has broad prospects in gene editing and molecular diagnostics. The CRISPR-Cas12 system can be used for double-stranded DNA detection and, once activated, exhibits trans-cleavage activity toward single-stranded DNA. The CRISPR-Cas13 system can be used for RNA detection and, once activated, exhibits trans-cleavage activity toward single-stranded RNA. Cas enzymes show relatively low trans-cleavage activity, resulting in limited sensitivity for detecting nucleic acids without pre-amplification, and often require coupling with isothermal amplification techniques to achieve optimal performance. Therefore, it is critical for the molecular diagnostics to enhance the trans-cleavage activity of the CRISPR-Cas system. However, to date, only a few studies have focused on optimizing Cas-based detection systems, and these approaches tend to be complex. Accordingly, there is an urgent need to develop a simple, feasible CRISPR-Cas system with high trans-cleavage activity to improve detection sensitivity.

Molecular chaperones are a class of helper proteins that assist intracellular protein folding, assembly, and transport, providing functions such as aiding the folding of unfolded proteins or nascent polypeptide chains, participating in protein trafficking and localization, preventing protein misfolding and aggregation, and repairing thermally denatured proteins. Current research on the molecular chaperones mainly focuses on the regulation of already folded proteins, and no literature reports involvement of molecular chaperones in modulating enzymatic activity of folded enzyme molecules.

### SUMMARY

The present invention aims to address the above-mentioned problems in the prior art and to provide a CRISPR-Cas system using a molecular chaperone to enhance trans-cleavage activity and an application thereof.

To achieve the above purpose, the technical solution of the present invention is as follows:

In a first aspect, the present invention provides a CRISPR-Cas system using a molecular chaperone to enhance trans-cleavage activity, and the CRISPR-Cas system includes: 1 pM-1 mM Cas enzyme, 1 pM-1 mM crRNA, 1*reaction buffer, 0.1 pM-1 mM target substrate, 1 nM-1 mM ssDNA reporter gene, and 1 pM-1 mM molecular chaperone.

The CRISPR-Cas system includes: 1 nM-1 μM Cas enzyme, 2 nM-2 μM crRNA, 1*reaction buffer, 1 pM-1 μM target substrate, 1 μM-100 μM ssDNA reporter gene, and 1 nM-100 μM molecular chaperone.

The Cas enzyme is Cas12a enzyme.

The molecular chaperone is ClpB, HSP70, Spy, or HSP20.

The Cas enzyme is Cas13a enzyme.

The molecular chaperone is Spy or HSP20.

The molecular chaperone is derived from a prokaryote, eukaryote, or archaeon.

The molecular chaperone is a recombinantly expressed protein or peptide fragment.

In a second aspect, the present invention provides an application of the above-mentioned CRISPR-Cas system in a molecular diagnostic.

In a third aspect, the present invention provides a kit, which includes the above-mentioned CRISPR-Cas system using a molecular chaperone to enhance trans-cleavage activity.

Compared with the prior art, the present invention has the following advantages:

The present invention provides a CRISPR-Cas system using a molecular chaperone to enhance trans-cleavage activity, which adds a molecular chaperone ClpB, HSP70, Spy, or HSP20 to the CRISPR-Cas system and enhances the trans-cleavage activity of the Cas enzyme to varying degrees, thereby improving detection sensitivity.

### BRIEF DESCRIPTION OF THE DRAWINGS

FIG. 1 shows relative NMR signal intensities of CRISPR-Cas12a without a molecular chaperone and with different molecular chaperones added.

FIG. 2 shows relative NMR signal intensities of CRISPR-Cas13a without a molecular chaperone and with different molecular chaperones added.

FIG. 3 shows fluorescence curves of CRISPR-Cas12a without a molecular chaperone and with different molecular chaperones added.

FIG. 4 shows fluorescence curves of CRISPR-Cas13a without a molecular chaperone and with different molecular chaperones added.

FIG. 5 shows trans-cleavage rates of CRISPR-Cas12a without a molecular chaperone and with different molecular chaperones added.

FIG. 6 shows trans-cleavage rates of CRISPR-Cas13a without a molecular chaperone and with different molecular chaperones added.

FIG. 7 shows limits of detection of CRISPR-Cas12a without a molecular chaperone and with the molecular chaperone HSP20 added.

FIG. 8 shows limits of detection of CRISPR-Cas13a without a molecular chaperone and with the molecular chaperone HSP20 added.

### DETAILED DESCRIPTION OF THE EMBODIMENTS

The present invention will be further described below in detail with reference to the drawings in combination with the embodiments.

### Main Materials and Instruments

TABLE 1
Main Materials and Instruments

| Reagents/Instruments | Model | Manufacturer |
|---|---|---|
| Lba Cas12a | — | New England Biolabs, |
| | | Inc. (NEB) |
| Lwa Cas13a | — | Magigen Biotechnology |
| | | Co., Ltd. |
| Protein Purification | NGC Quest | Bio-Rad Company |
| System | 10plus | |
| Real-Time Quantitative | QuantStudio 3 Real- | Thermo Scientific |
| PCR Instrument | Time PCR System | Company |
| PH Meter | FiveEasy Plus | METTLER Company |
| Electrophoresis | 164-5050 | Bio-Rad Company |
| Apparatus | | |
| Benchtop Centrifuge | FRESCO17 | Thermo Scientific |
| | | Company |
| NanoDrop ™ One/One^{C} | 8400-317400 | Thermo Scientific |
| Ultramicro UV | | Company |
| Spectrophotometer | | |

### Embodiment 1 Comparison of Relative NMR Signal Intensity

The relative NMR signal intensity of the Cas enzyme is measured in the absence or presence of different molecular chaperones. The NMR reaction system without the molecular chaperone includes 50 μM Cas12a or Cas13a enzyme, 20 mM NaP, pH 7.4, and 1000 D₂O. The NMR reaction system with the molecular chaperone includes 50 μM Cas12a or Cas13a enzyme, 100 μM molecular chaperone, 20 mM NaP, pH 7.4, and 10% D₂O. The molecular chaperone is selected from ClpB derived from thermophiles, HSP70 derived from mammals, Spy derived from bacteria, and HSP20 derived from archaea. The molecular chaperone is obtained as follows: expression vectors carrying ClpB, HSP70, Spy, and HSP20 (synthesized by Shenggong Bioengineering Co., Ltd.; NCBI accession numbers: ClpB WP_011228712.1, HSP70 KFO35963.1, Spy HCO1701554.1, HSP20 WP_048053707.1) are transformed into Escherichia coli (E. coli) cells. After lysis of the recombinant cells, the recombinant proteins ClpB, HSP70, Spy, and HSP20 are initially purified by affinity chromatography and then further purified by molecular sieve refined purification to obtain purified ClpB, HSP70, Spy, and HSP20 proteins.

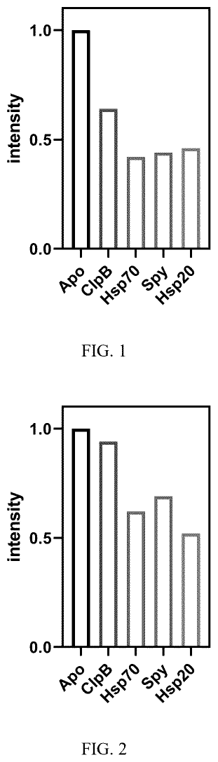

The relative NMR signal intensity detection results of Cas12a enzyme are shown in FIG. 1, and the relative NMR signal intensity detection results of Cas13a enzyme are shown in FIG. 2, in which the Apo group represents the group without the molecular chaperone. From FIG. 1, it can be seen that the relative NMR signal intensities of Cas12a enzyme after adding different molecular chaperones are all significantly lower than those of Cas12a enzyme without the molecular chaperone. This is because the molecular chaperones ClpB, HSP70, Spy, and HSP20 can all bind to Cas12a enzyme to form larger complexes, resulting in a decrease in NMR signal intensity. From FIG. 2, it can be seen that the relative NMR signal intensities of Cas13a enzyme after adding the molecular chaperones HSP70, Spy, or HSP20 are significantly lower than those of Cas13a enzyme without the molecular chaperone, whereas the relative NMR signal intensity of the CRISPR-Cas13a system with the molecular chaperone ClpB is similar to that without the molecular chaperone. This indicates that HSP70, Spy, and HSP20 can bind to Cas13a enzyme to form larger complexes, resulting in a decrease in NMR signal intensity, while ClpB may not interact with Cas13a enzyme.

### Embodiment 2 Comparison of Trans-Cleavage Rates

The fluorescence curves of the CRISPR-Cas12a/Cas13a systems are measured in the absence or presence of different molecular chaperones.

The reaction procedure of the Cas12a system is as follows: in a 200 μL PCR tube, 2 μL of 100 nM Cas12a, 2 μL of 200 nM crRNA, 2 μL of 10*reaction buffer, and 8 μL of RNase-free H₂O are successively added and incubated at 37° C. for 15 min; and 2 μL of 1 μM molecular chaperone, 2 μL of 10 nM dsDNA target substrate, and 2 μL of 2.5 μM ssDNA reporter gene are added, bringing the final volume to 20 μL. The molecular chaperone is selected from ClpB, HSP70, Spy, and HSP20. The fluorescence intensity is recorded every 30 seconds at 37° C. using a real-time quantitative PCR instrument.

The reaction procedure of the Cas13a system is as follows: in a 200 μL PCR tube, 1 μL of 1 μM Cas13a, 1 μL of 1 μM crRNA, 1 μL of 40 U/μL RNase inhibitor, 5 μL of 10*reaction buffer, and 12 μL of RNase-free H₂O are successively added and incubated at 37° C. for 10 min; and 5 μL of 1 μM molecular chaperone, 5 μL of 10 nM RNA target substrate, and 20 μL of 1 μM ssRNA reporter gene are added, bringing the final volume to 50 μL. The molecular chaperone is selected from ClpB, HSP70, Spy, and HSP20. The fluorescence intensity is recorded every 30 seconds at 37° C. using a real-time quantitative PCR instrument.

TABLE 2
Sequence information

| Enzyme | Nucleic | |
|---|---|---|
| Type | Acid Type | Base Sequence |
| Cas12a | crRNA | 5′-UAAUUUCUACUAAGUGUAGAUGUCUAGCUACAGAGAAA-3′ |
| | | (SEQ ID NO: 1) |
| | dsDNA | 5′-AATAGGTGATTTTGGTCTAGCTACAGAGAAATCTCGATG-3′ |
| | Target | (SEQ ID NO: 2) |
| | Substrate | 3′-TTATCCACTAAAACCAGATCGATGTCTCTTTAGAGCTAC-5′ |
| | | (SEQ ID NO: 3) |
| | ssDNA | 5′6-FAM-TTTTTTTTTTTTTTT-3′BHQ1 (SEQ ID NO: 4) |
| Cas13a | crRNA | 5′-GAUUUAGACUACCCCAAAAACGAAGGGGACUAAAACAC |
| | | ACUACCUGCACUAUAAGCACUUUAGUGC-3′ (SEQ ID NO: 5) |
| | RNA | 5′-GUAGCACUAAAGUGCUUAUAGUGCAGGUAGUGUUUA-3′ |
| | Target | (SEQ ID NO: 6) |
| | Substrate | |
| | ssRNA | 5′6-FAM-MAAUGGCMA-3′BHQ1 (SEQ ID NO: 7) |

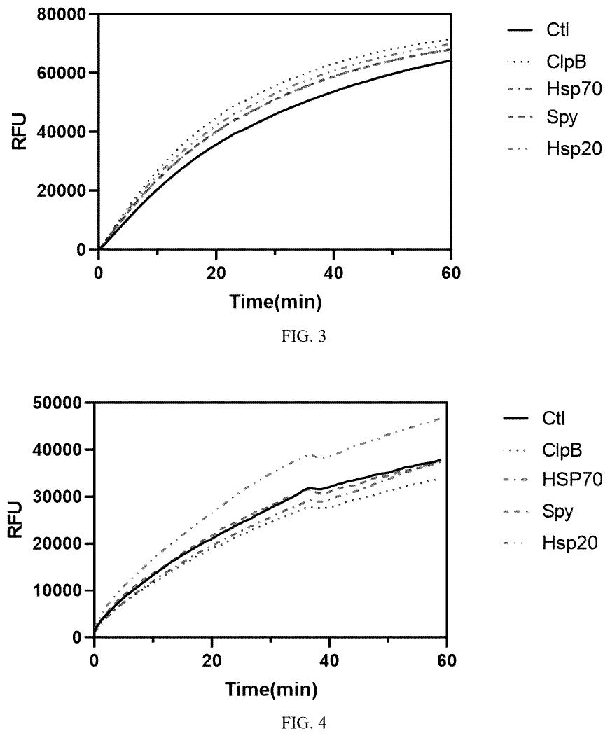

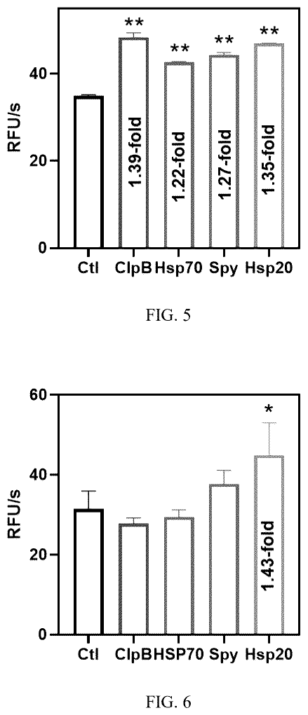

The fluorescence curve of the Cas12a system is obtained as shown in FIG. 3, and the fluorescence curve of the Cas13a system is obtained as shown in FIG. 4, and the Ctl group represents a control group without the molecular chaperone. Based on FIG. 3 and FIG. 4, the trans-cleavage rates of the CRISPR-Cas12a/Cas13a systems in the absence or presence of different molecular chaperones are calculated. The calculated trans-cleavage rate of the Cas12a system is shown in FIG. 5, and the calculated trans-cleavage rate of the Cas13a system is shown in FIG. 6, and the Ctl group represents a control group without the molecular chaperone. As shown in FIG. 5, the trans-cleavage rates of the Cas12a system with different molecular chaperones are significantly increased compared with that of the Cas12a system without the molecular chaperone. As shown in FIG. 6, the trans-cleavage rates of the Cas13a system with the molecular chaperone HSP20 or Spy are markedly increased compared with that of the Cas13a system without the molecular chaperone, whereas the fluorescence curve of the Cas13a system with the molecular chaperone ClpB or HSP70 remains essentially unchanged relative to the fluorescence curve of the Cas13a system without the molecular chaperone.

### Embodiment 3 Comparison of Limits of Detection (LOD)

The trans-cleavage rates of the CRISPR-Cas12a/Cas13a systems without the molecular chaperone or with the molecular chaperone HSP20 added are measured at target substrate concentrations of 0 pM, 3 pM, 10 pM, 30 pM, 100 pM, 300 pM, and 1000 pM, and the LOD is calculated using the formula LOD=3σ/K.

The reaction procedure of the Cas12a system is as follows: in a 200 μL PCR tube, 2 μL of 100 nM Cas12a, 2 μL of 200 nM crRNA, 2 μL of 10*reaction buffer, and 8 μL of RNase-free H₂O are successively added and incubated at 37° C. for 15 min; and 2 μL of 1 μM HSP20, 2 μL of the dsDNA target substrate at each specified concentration, and 2 μL of 2.5 μM ssDNA reporter gene are added, bringing the final volume to 20 μL. The fluorescence intensity is recorded every 30 seconds at 37° C. using a real-time quantitative PCR instrument.

The reaction procedure of the Cas13a system is as follows: in a 200 μL PCR tube, 1 μL of 1 μM Cas13a, 1 μL of 1 μM crRNA, 1 μL of 40 U/μL RNase inhibitor, 5 μL of 10*reaction buffer, and 12 μL of RNase-free H₂O are successively added and incubated at 37° C. for 10 min; and 5 μL of 1 μM HSP20, 5 μL of the RNAtarget substrate at each specified concentration, and 20 μL of 1 μM ssRNA reporter gene are added, bringing the final volume to 50 μL. The fluorescence intensity is immediately recorded every 30 seconds at 37° C. using a real-time quantitative PCR instrument.

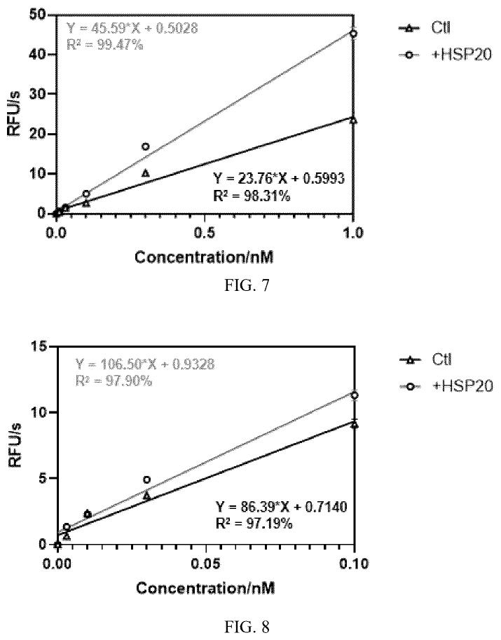

Based on the Cas12a fluorescence curve, the LOD of the Cas12a system is shown in FIG. 7, and based on the Cas13a fluorescence curve, the LOD of the Cas13a system is shown in FIG. 8, and the Ctl group represents a control group without the molecular chaperone. As shown in FIG. 7, the addition of the molecular chaperone HSP20 increases the LOD of the Cas12a system by 1.8-fold, indicating that the molecular chaperone HSP20 can enhance the sensitivity of the CRISPR-Cas12a system in molecular diagnostics. As shown in FIG. 8, the addition of the molecular chaperone HSP20 increases the LOD of the Cas13a system by 1.2-fold, indicating that the molecular chaperone HSP20 can enhance the sensitivity of the CRISPR-Cas12a system in molecular diagnostics.

### Embodiment 4

A kit includes the CRISPR-Cas system as described in the Embodiment 2.

What is claimed is:

1. A CRISPR-Cas system using a molecular chaperone to enhance trans-cleavage activity, comprising: 1 pM-1 mM Cas enzyme, 1 pM-1 mM crRNA, 1*reaction buffer, 0.1 pM-1 mM target substrate, 1 nM-1 mM ssDNA reporter gene, and 1 pM-1 mM molecular chaperone.

2. The CRISPR-Cas system using the molecular chaperone to enhance trans-cleavage activity according to claim 1, wherein the CRISPR-Cas system comprises: 1 nM-1 μM Cas enzyme, 2 nM-2 μM crRNA, 1*reaction buffer, 1 pM-1 μM target substrate, 1 μM-100 μM ssDNA reporter gene, and 1 nM-100 μM molecular chaperone.

3. The CRISPR-Cas system using the molecular chaperone to enhance trans-cleavage activity according to claim 1, wherein the Cas enzyme is Cas12a enzyme.

4. The CRISPR-Cas system using the molecular chaperone to enhance trans-cleavage activity according to claim 3, wherein the molecular chaperone is ClpB, HSP70, Spy, or HSP20.

5. The CRISPR-Cas system using the molecular chaperone to enhance trans-cleavage activity according to claim 1, wherein the Cas enzyme is Cas13a enzyme.

6. The CRISPR-Cas system using the molecular chaperone to enhance trans-cleavage activity according to claim 5, wherein the molecular chaperone is Spy or HSP20.

7. The CRISPR-Cas system using the molecular chaperone to enhance trans-cleavage activity according to claim 4, wherein the molecular chaperone is derived from a prokaryote, eukaryote, or archaeon.

8. The CRISPR-Cas system using the molecular chaperone to enhance trans-cleavage activity according to claim 4, wherein the molecular chaperone is a recombinantly expressed protein or peptide fragment.

9. A molecular diagnostic method, comprising using the CRISPR-Cas system using the molecular chaperone to enhance trans-cleavage activity according to claim 1.

10. A kit, comprising the CRISPR-Cas system using the molecular chaperone to enhance trans-cleavage activity according to claim 1.

11. The CRISPR-Cas system using the molecular chaperone to enhance trans-cleavage activity according to claim 6, wherein the molecular chaperone is derived from a prokaryote, eukaryote, or archaeon.

12. The CRISPR-Cas system using the molecular chaperone to enhance trans-cleavage activity according to claim 6, wherein the molecular chaperone is a recombinantly expressed protein or peptide fragment.

13. The molecular diagnostic method according to claim 9, wherein the CRISPR-Cas system comprises: 1 nM-1 μM Cas enzyme, 2 nM-2 μM crRNA, 1*reaction buffer, 1 pM-1 μM target substrate, 1 μM-100 μM ssDNA reporter gene, and 1 nM-100 μM molecular chaperone.


SEQUENCE LISTING (6 sequences)

SEQ ID NO: 1 - RNA, 38 nt, unidentified
  source 1..38: mol_type = other RNA
taatttctactaagtgtagatgtctagctacagagaaa

SEQ ID NO: 2 - DNA, 39 bp, unidentified
  source 1..39: mol_type = other DNA
aataggtgattttggtctagctacagagaaatctcgatg

SEQ ID NO: 3 - DNA, 39 bp, unidentified
  source 1..39: mol_type = other DNA
ttatccactaaaaccagatcgatgtctctttagagctac

SEQ ID NO: 4 - DNA, 15 bp, synthetic construct
  source 1..15: mol_type = other DNA
ttttttttttttttt

SEQ ID NO: 5 - RNA, 66 nt, unidentified
  source 1..66: mol_type = other RNA
gatttagactaccccaaaaacgaaggggactaaaacacactacctgcactataagcactttagtgc

SEQ ID NO: 6 - RNA, 36 nt, unidentified
  source 1..36: mol_type = other RNA
gtagcactaaagtgcttatagtgcaggtagtgttta